(19) United States
(12) Patent Application Publication
(10) Pub. No.: US 2005/0147650 A1
(43) Pub. Date: Jul. 7, 2005

(54) Feed for silkworm, silk produced from silkworms that feed on the feed for silkworm, and silk products made from the silk

(76) Inventor: Naoto Kigasawa, Gumma (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/751,526

(22) Filed: Jan. 6, 2004

(51) Int. Cl. A23K 1/165; A23K 1/17; A01K 67/04; A23K 1/00

(52) U.S. Cl. 424/442; 426/635

(57) ABSTRACT

Silk manufactured from silkworms reared with feed containing a pre-determined quantity of organic or inorganic particles, an aqueous solution of a pre-determined concentration of organic or inorganic particles, or a distillate such as bamboo vinegar, which is mixed in, added in, sprayed on or coated on artificial silkworm feed or fresh mulberry leaves. Silk manufactured from silkworms reared with feed containing a mixture or addition of a pre-determined quantity of functional particles, an aqueous solution of functional particles of pre-determined concentration, or a distillate such as bamboo vinegar, wherein said functional particles are alumina, silica, titanium oxide, zeolite, fluorite (luminous rock), tourmaline, jade, pigment, dye, temperature-sensitive pigment, UV-sensitive pigment or any other functional pigments, perfume, charcoal, cholesterol or other inorganic or organic substances.

### TECHNICAL FIELD OF THE INVENTION

The present invention relates to silkworm feed that consists of artificial silkworm feed or fresh mulberry leaves to which at least one of a pre-determined quantity of functional particles, an aqueous solution of functional particles of a pre-determined concentration, and distillates such as bamboo vinegar are mixed or added. The present invention further relates to silk produced from silkworms that feed on the silkworm feed and silk products made from the silk.

### BACKGROUND OF THE INVENTION

Conventional methods are available for coating threads and fabric materials with functional particles to provide additional functionality. Clothing and other products have been developed using these materials to impart these finished goods with such new functionality.

### SUMMARY OF THE INVENTION

Coating threads or fabrics with functional particles causes a loss of the natural texture and touch of silk. The particles may come off when the materials are washed. At present, products using functional particles that closely maintain the natural texture and touch of silk have not been commercialized. The inventors of the present invention have bred silkworms with feed containing inorganic substances that are considered most inadequate as the content of silkworm feed. The derived data disprove the established theory that the inorganic substances eaten by silkworms will not transfer to the silk that they produce.

The present invention is based on this epoch-making knowledge. According to the present invention, functional particles of inorganic substances considered inadequate for use in silkworm feed are mixed in the feed and the inorganic substances are transferred to the cocoon filaments in a natural form through the silkworms. It is therefore easy to produce silk with added functionality which is very close to the natural texture and touch that has charmed humans since early times. The functional particles may include alumina, silica, titanium oxide, zeolite, fluorite (luminous rock), tourmaline, jade and other minerals, pigment, dye, aromatics, temperature-sensitive pigments, UV-sensitive pigments and other functional pigments, perfume, charcoal, cholesterol and other inorganic and organic substances which silkworms eat.

Clothing (such as fabrics and knits) and non-clothing (such as fish lines) with new functionality may be produced by knitting or weaving the functional silk containing the functional particles with other fibers (wool, hemp, cotton, synthetic fiber, etc.). When using silk of the present invention together with other fabrics, a part of the product may have a peculiar functionality. Partial use of the functional silk may be used to enhance the local functions of a product (for example, infrared ray-generating silk may be used at the shoulder portion of a shirt so that the wearer's body warmth is retained at the shoulders).

The present invention was developed in consideration of the above circumstances and intends to solve the problems of conventional methods by providing a novel silkworm feed that contains at least one of a pre-determined quantity of functional particles, aqueous solution of functional particles of a pre-determined concentration, and distillates such as bamboo vinegar. The feed for a silkworm of the present invention may include an artificial silkworm feed, in particular, it may be one known as “KUWANOHANA.” The aqueous solution of the functional particles may contain at least 0.5 to 40% of the functional particles by weight, even though the amount may be less than that. The artificial silkworm feed may preferably contain at least 1 to 30% of the functional substances by weight, even though the amount may be less than that. The functional particles may be any one of or two or more of alumina, silica, titanium oxide, zeolite, fluorite (luminous rock), tourmaline, jade, pigment, dye, temperature-sensitive pigment, UV-sensitive pigment or any other functional pigments, perfume, charcoal, cholesterol and other inorganic or organic substance. The functional particles may preferably powder particles of 10 μm or less in diameter, even though the size may be more than that. The feed may include fresh mulberry leaves.

The present invention also provides silk and silk products produced from silkworms, to which silkworm feed of the present invention is fed. The silk products of the present invention may be made from the silk only or made from a combination of the silk and other fiber or fibers.

The present invention makes it is easily possible to produce cocoon filaments containing functional particles by mixing functional particles, which are conventionally considered inadequate for use in silkworm feed, in the artificial feed, mulberry leaves or other feed and feeding the mixture to the silkworms. The functional particles contained in clothing made from the cocoon filaments provide benefits for humans. It is also possible to easily produce clothing containing various functional particles. Silk of the present invention can be used for various products and is not limited to clothing. It is possible to produce clothing and other products by combining functional silk produced by the present invention with other fibers (wool, hemp, cotton, synthetic fibers, etc.).

### BRIEF DESCRIPTION OF THE DRAWINGS

FIG. 1 is a photograph of cocoon filaments viewed under a scanning electron microscope (SEM) (×300).

FIG. 2 is a photograph of cocoon filaments viewed under a scanning electron microscope (SEM) (×300);

FIG. 3 is a graph showing energy dispersion of silk containing jade.

FIG. 4 is a photograph of cocoon filaments containing zeolite particles viewed under a scanning electron microscope (SEM).

FIG. 5 is a graph of far-infrared emissivity of bed sheet made of 10% zeolite cocoon filament and 90% wool.

FIG. 6 is a graph showing minus ions emitted by a knit product (wristband) made of silk from cocoon filaments containing tourmaline and cotton.

### DETAILED DESCRIPTION OF THE INVENTION

The first working example of the present invention is a silkworm feed wherein the functional particles are mixed in artificial silkworm feed or fresh mulberry leaves. Functional particles are mixed in the feed by directly spraying or mixing them with the feed or by preparing aqueous solutions of functional particles and spraying or coating such solutions onto the feed. The functional particles may be alumina, silica, titanium oxide, zeolite, fluorite (luminous rock), tourmaline, jade and other minerals, pigment, dye, perfume, temperature-sensitive pigment, UV-sensitive pigment and any other functional pigments, aromatics, charcoal, cholesterol or other inorganic or organic substances which are edible by silkworms. Distillates such as bamboo vinegar may be used instead of the functional aqueous solutions. The second working example is silk produced from silkworms that feed on the silkworm feed. The third working example is silk products produced by knitting or weaving the silk. The fourth working example is silk products produced by knitting or weaving the silk together with other fibers.

Silkworm feed containing functional particles of the present invention (the first working example) is described below. Two methods are available to feed the functional particles to silkworms. The first method is to mix or add a pre-determined quantity of functional particles to artificial silkworm feed or fresh mulberry leaves. The second method is to spray or coat aqueous solutions containing the functional particles onto artificial silkworm feed or fresh mulberry leaves.

The functional particles are defined in this specification to include alumina, silica, titanium oxide, zeolite, fluorite (luminous rock), tourmaline, jade and other minerals, pigment, dye, aromatics, temperature-sensitive pigment, UV-sensitive pigment or any other functional pigments, perfume, charcoal, and cholesterol or other organic or inorganic substances which are edible by silkworms. Distillates such as bamboo vinegar may be used instead of the functional aqueous solutions. Experiments have shown that the functional substances will be absorbed or attach to fibroin when distilled water or other distillates are used.

The first method (mixing or adding a pre-determined quantity of functional particles to artificial silkworm feed or fresh mulberry leaves) is described below. The method of mixing functional particles with the powder of KUWANOHANA (trade name of a product of Nosan Corporation, Gunnma, Japan, the trademark is registered in Japan), a popular artificial silkworm feed distributed throughout Japan, is described below. (See Table 1 for the composition of the feed.)

When using the KUWANOHANA artificial feed, at least one kind of functional particle with a particle size similar to or smaller than the KUWANOHANA powder particles is mixed in a pre-determined quantity and water is added. The mixture is steamed to prepare a wet feed containing functional particles. The prepared feed is given to the silkworms to produce cocoons containing functional particles. The cocoons are used to produce silk containing functional particles. The artificial feed is not limited to KUWANOHANA but may be any other artificial silkworm feed.

Artificial Feed Containing Functional Particles

The composition of the KUWANOHANA (registered trademark) artificial feed is shown in Table 1. Aluminum, silica, barium, titanium oxide, zeolite, tourmaline or other ceramics, luminous rock (fluorite), organic substances (cholesterol, etc.) and at least one of the substances considered inadequate as feed for silkworms other than ceramics, with a particle size the same or less than the size of KUWANOHANA powder particles, is added to the artificial feed or fresh mulberry leaves in a ratio of 1 to 25% by dry weight of the feed or leaves. The mixing ratio represents the quantity of functional particles up to which silkworms will not reject eating the feed. The ratio was determined through experience by feeding variable quantities of functional particles to silkworms. This ratio is preferably 1 to 25%, or more preferably, 1 to 10%. Water is then dded in an amount approximately 2.5 times the material by weight, the mixture is heated for disinfection, packed and neatly formed in vinyl bags and refrigerated for preservation until use.

TABLE 1
Composition of Artificial Feed Used in Experiments

| Raw Materials | Mixing Ratio |
|---|---|
| Powder of dry mulberry leaves | 30.0% |
| Defatted soybean powder | 28.0 |
| Cellulose | 15.0 |
| Forming agent | 7.0 |
| Starch | 5.1 |
| Sugar | 4.0 |
| Inorganic salt mixture | 4.0 |
| Citric acid | 3.5 |
| Vitamin C | 0.5 |
| Vitamin B mixture | 0.4 |
| Soybean oil | 1.4 |
| Antiseptic | 1.1 |
| Total | 100.0 |

Composition of KUWANOHANA feed for the 1^{st} instar

Typical examples of the functions of the above materials are described below.
Alumina: Generates far-infrared rays.
Silica: Provides UV-blocking function.
Titanium oxide: Provides an antibacterial feature.
Tourmaline: Generates minus ions that have a favorable effect on the body.
Charcoal: Eliminates odor.
Jade: Generates ultrasonic waves that have a favorable effect on the body.
Carbon: Makes silk electrically conductive.
Zeolite: Generates far-infrared rays, controls bacteria, and eliminates odor.
Fluorite (luminous rock): Accumulates light (glows in the dark).
Cholesterol: Invigorates the skin.
Pigment and dye: Enables coloring.
Aromatics and perfume: Provides fragrance.
Temperature-sensitive pigment: Color of the silk changes according to temperature.
UV-sensitive pigment: Color of the silk changes under ultraviolet rays (sunlight).
Bamboo Vinegar: Eliminates odor, controls bacteria.

Silk derived from silkworms will have the above functions when the functional particles are mixed in the silkworm feed. One type of the functional particles are basically mixed into each batch of feed but it is possible to mix two or more types of functional particles in the same batch of feed.

The size of the functional particles mixed into the feed is preferably no more than 10 μm (the size up to which silkworms will not reject eating the feed). If the particle size is too large, the silkworms tend not to eat the functional particles. The mixing ratio and particle size which silkworms will not reject are easily determined in experiments by gradually increasing the particle ratio and particle size starting from zero. The above figures were determined in this way through experiments.

This prepared feed is fed to the silkworms and the silk they produce is used to produce products. Generally, just one type of functional particle is used in the feed to produce silk of a single functionality. To produce silk products of diverse functionality, more than one type of silk with different functionalities may be combined by knitting or weaving rather than using the same type of silk. It is also possible to combine other fibers with such silk. This enables a single product to have more than one function.

The flow of silkworm feed manufacture using ceramics as functional particles is shown below.
Composition of KUWANOHANA artificial silkworm feed+Mix ceramic particles→Add waters→Knead→Steam or boil→Bag→Form→Refrigerate→Feed

Steam or boil means steaming or boiling the wet feed. The formed artificial feed is bagged, stored in a refrigerator to preserve it, and used for feeding silkworms as appropriate.

Feeding Silkworms with Feed Containing Ceramics

With the objective being the inclusion of ceramics in the cocoon filaments, the inventors studied the best time in the larval stage of the silkworms to feed them feed containing ceramics from an economical point of view. The inventors also studied the relationship between quantity of ceramics included in the feed and the impact on the silkworms. The standard feed rate for silkworms in each instar is shown in Table 2. Since ceramics have little nutritional value, they are added to each feed batch. The elements of the ceramics mixed in the artificial silkworm feed are, for example, Ti, Ba, O, Al and Si. The particle size is 10 μm or less. The quantity of fresh mulberry leaves fed to the silkworms is increased 20% by weight in anticipation of drying. The silkworms ate the artificial feed containing ceramics with no difference from the conventional artificial feed at the ordinary breeding temperature and atmosphere.

TABLE 2
Quantity of Feed and Rearing Temperatre
by Instar (for 20,000 silkworms)

| Item | 1^{st} Instar | 2^{nd} Instar | 3^{rd} Instar | 4^{th} Instar | 5^{th} Instar |
|---|---|---|---|---|---|
| Quantity of | 1 kg | 3 kg | 13 kg | 34 kg | 212 kg |
| Feed | | | | | |
| Rearing | 29° C. | 28° C. | 27° C. | 25° C. | 24° C. |
| Temperature | | | | | |

Results of Feeding and Observation

(a) Experiment 1: Table 3 shows the results of feeding the artificial silkworm feed containing ceramics during the 2nd instar through mounting. As the table shows, the pupation rate (number of pupas that produced a cocoon and became a pupa in the experiment) did not differ significantly for both male and female silkworms fed feed with or without the addition of ceramics. The mass of cocoons, mass of cocoon shells and cocoon shell yield were better for silkworms fed the feed containing ceramics.

TABLE 3
Results of Feeding Ceramics in 2^{nd} Instar through Mounting

| | | | Mass of | Cocoon | |
|---|---|---|---|---|---|
| Quantity of | Rate of | Mass of | Cocoon | Shell | Result of |
| Ceramics | Pupation | Cocoon | Shell | Yield | Observation |
| Comparison | ♀ 98% | 1.73 g | 34.3 mg | 19.8% | None |
| | ♂ 98% | 1.35 g | 28.8 mg | 21.3% | None |
| | M 98% | 1.54 g | 316 mg | 20.5% | None |
| 10% | ♀ 100% | 1.98 g | 39.2 mg | 19.8% | Yes |
| | ♂ 100% | 1.39 g | 30.5 mg | 21.3% | Yes |
| | M 100% | 1.69 g | 34.9 mg | 20.6% | Yes |

* Rate of pupation: Number of silkworms that spun a cocoon and became a pupa in the experiment.

(b) Experiment 2: Table 4 shows the results of feeding the artificial silkworm feed containing ceramics during the 4th instar through mounting. As the table shows, there was no change in the pupation rate (number of pupas that produced a cocoon and became a pupa in the experiment) when the quantity of ceramics in the feed was increased (pupation rate was lower for feed not containing ceramics). The mass of cocoons, mass of cocoon shells and cocoon shell yield were better for silkworms fed the feed containing ceramics.

TABLE 4
Results of Feeding Ceramics in 4th Instar through Mounting

| | | | Mass of | Cocoon | |
|---|---|---|---|---|---|
| Quantity of | Rate of | Mass of | Cocoon | Shell | Result of |
| Ceramics | Pupation | Cocoon | Shell | Yield | Observation |
| 0% | 98% | 1.73 g | 34.3 mg | 19.1% | None |
| 1 | 100 | 1.85 | 37.2 | 20.1 | Yes |
| 2.5 | 100 | 1.84 | 35.9 | 19.5 | Yes |
| 5 | 100 | 1.98 | 39.2 | 20.8 | Yes |
| 10 | 100 | 1.98 | 39.2 | 19.8 | Yes |
| 15 | 100 | 1.89 | 39.7 | 21.0 | Yes |
| 25 | 100 | 1.69 | 34.9 | 20.6 | Yes |

(c) Experiment 3: Table 5 shows the results of feeding the artificial silkworm feed containing ceramics during the 5th instar through mounting.

TABLE 5
Results of Feeding Ceramics in 5th Instar through Mounting

| Quantity of Ceramics | Pupation | Mass of | Mass of | Cocoon | Result of |
|---|---|---|---|---|---|
| and timing of feeding | (%) | Cocoon | Cocoon Shell | Shell (%) | Observation |
| 0: Start of 5th instar | 98% | 1.83 g | 37.0 mg | 20.2% | None |
| 10%: Start of 5th instar | 100 | 1.98 | 41.8 | 21.1 | Yes |
| 5%: Day 4, 5th instar | 100 | 1.94 | 40.5 | 20.9 | Yes |

In all of the three experiments above, the silkworms grew healthily throughout the periods while eating artificial feed containing ceramics. The mass of cocoons and cocoon shells was heavier than when the silkworms ate feed without ceramics. The cocoon shell yield was also higher. No discernible relationship was found with the ratio of ceramics in the mixture.

The above describes the case where ceramics are mixed with artificial feed. To mix ceramics with fresh mulberry leaves, a pre-determined quantity of ceramics is sprayed on fresh mulberry leaves, which are then fed to the silkworms. Spraying is performed several times so that the ceramics are attached to the leaves uniformly. A slightly smaller quantity of this mulberry leaf feed than usually given is fed to the silkworms so that all the feed is eaten.

Summary of Silkworm Breeding with Artificial Feed

A pre-determined amount of feed containing ceramics was fed to the silkworms. They did not reject the feed but ate it well. The size of the silkworms was more uniform, and their health showed improvement. In particular, the silkworms grew steadily and produced cocoons when they ate feed containing ceramics in a ratio of 25% by dry weight. This verifies that ceramics are harmless to the silkworms. The quantity of feed eaten by the silkworms until they spun a cocoon was greater where more ceramics were included in the feed. The quantity of feed eaten was roughly proportional to the quantity of ceramics included.

The second method of feeding functional particles to silkworms is described below. An aqueous solution was prepared by mixing functional particles and this particle solution was sprayed or coated on the artificial silkworm feed or fresh mulberry leaves. Functional particles were mixed in clean water at a ratio of 0.5 to 50% by weight to prepare the functional aqueous solutions. The mulberry leaves were sprayed with this solution or immersed in it. For the artificial silkworm feed, the aqueous solutions containing functional particles were added by spraying or coating them onto the feed. These types of feed should preferably be used when the water content has just started drying.

Table 6 shows the results of breeding silkworms by feeding mulberry leaves sprayed with ceramic solutions.

TABLE 6
Results of Rearing: Ceramic Powder Sprayed on Mulberry Leaves

| per 1 kg of | Mass of | Mass of | Cocoon | Result of |
|---|---|---|---|---|
| mulberry Leaves | Cocoon | Cocoon Shell | Shell Yield | Observation |
| 0 g | 1.89 g | 42.1 mg | 22.3% | None |
| 5 | 1.91 | 42.6 | 22.3 | Yes |
| 20 | 1.98 | 44.6 | 22.5 | Yes |

Table 7 shows the results of breeding silkworms by feeding mulberry leaves immersed in ceramic solutions.

TABLE 7
Results of Rearing: Mulberry Leaves Immersed in Ceramic Solutions

| per 1 liter of | Mass of | Mass of | Cocoon | Result of |
|---|---|---|---|---|
| mulberry Leaves | Cocoon | Cocoon Shell | Shell Yield | Observation |
| 0 g | 1.89 g | 42.1 mg | 22.3% | None |
| 20 g | 1.97 | 44.1 | 22.4 | Yes |
| 300 g | 2.11 | 49.9 | 22.6 | Yes |

Summary of Silkworm Breeding with Mulberry Leaves Containing Ceramics

In both cases of mulberry leaves sprayed with ceramics or immersed in ceramic solutions, the silkworms ate well and spun a cocoon in the same way as when they were fed with artificial feed containing ceramics in all sections. Microscopic observation verified the transfer of the ceramics to the cocoon filaments.

Confirmation of the transfer of ceramics from silkworms grown with the above artificial feed or mulberry leaves containing ceramics to the cocoon filaments by scanning electron microscope. The transfer of ceramic particles to cocoon filaments was confirmed under a scanning electron microscope with 300 to 1000 magnification power. The elements present in the ceramics were analyzed using an energy dispersive x-ray analyzer.

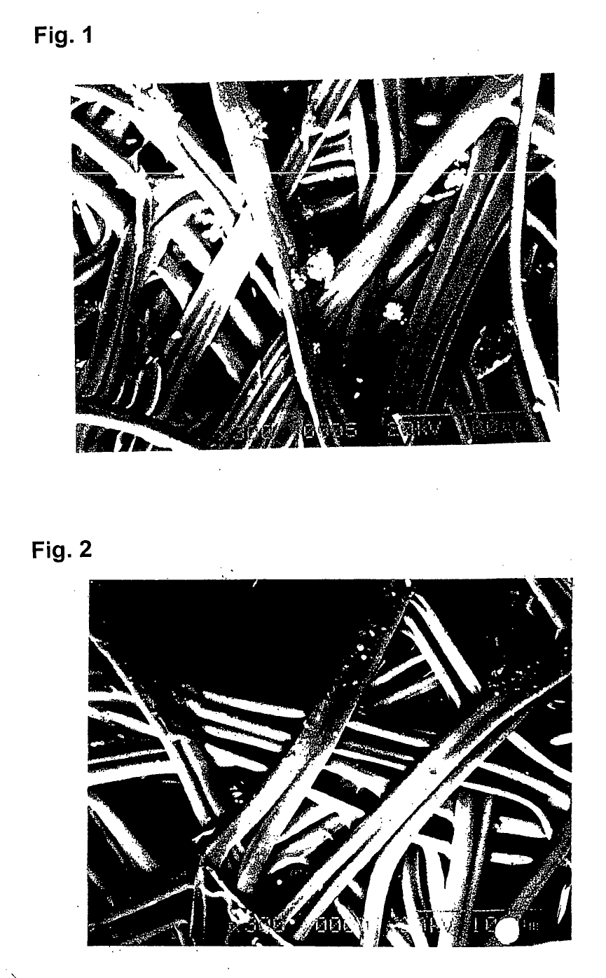

FIGS. 1 and 2 are cocoon filaments photographed under a scanning electron microscope (SEM). The white dots are ceramics transferred to the filaments. Ceramic transfer to the filaments was confirmed in all three experiments of rearing silkworms with the feed of the present invention: Experiment 1 where the feed was given from the 2nd instar (young silkworms), Experiment 2 where the feed was given from the 4th instar (middle stage silkworm period), and Experiment 3 where the feed was given from the early stage of the 5th (final) instar. Ceramic transfer to the cocoon filaments was confirmed in all three experiments.

These results suggest that the most economical timing for starting feeding with ceramics is the 4th day in the 5th instar when the silk gland grows sharply (Experiment 3). The ceramic content was intentionally varied between 1 and 25%. In all the cases, it was clear that the ceramics were taken in the cocoon filaments. Microscopic observation revealed that ceramic deposits were relatively large on sericin (surface of silk). When distillates such as bamboo vinegar were used, it was confirmed that functional particles were included in or attached to fibroin.

Regarding concentration of functional particles in the feed, the amount of functional particles transferred to the cocoon filaments slightly increased with larger amounts of particles added to the feed. The transfer of functional particles to the cocoon filaments was studied for both male and female silkworms, and there was no difference noted between them. This indicates that the most efficient method of supplying feed containing functional particles is in the 5th instar period when the silk gland develops.

Microscopic observation revealed that a large portion of the ceramics migrated to sericin. In this light, it is urgently necessary to develop methods to use cocoon filaments without removing sericin. The raw filament before removing sericin is generally called raw silk which shows “Shari”, meaning that it is hard and elastic. Silk has been popular as a raw material for knitwear in recent years. Silk for knitwear does not require refinement to remove sericin (such as that required for producing conventional Japanese clothing). Furthermore, “Shari” (hard and elastic filaments) including sericin is increasingly used in outerwear. Based on these recent trends, the development of new knitwear using raw filaments containing sericin is expected through a combination of the two methods. Raw filaments contain sericin in a quantity equal to approximately 25% of the filament by weight. This method of using sericin as it is contained in the raw silk is both economical and epoch-making because it saves 25% of the raw material compared with using fibroin only after refinement to produce Japanese clothing.

The present invention, as described in detail in the above paragraphs, is based on the epoch-making discovery of the fact that disproves the established theory that any inorganic substances eaten by silkworms will not transfer to the silk they produce. According to the present invention, feed containing functional particles is prepared and fed to the silkworms and silk threads containing these functional particles can be produced easily. It is also possible to produce silk containing functional particles that is very close in texture and feeling to the charm of natural silk.

The KUWANOHANA feed was used in the experiments on artificial feed containing ceramics. Various other types of artificial feed available in the market may also produce similar silk when they are mixed with functional particles.

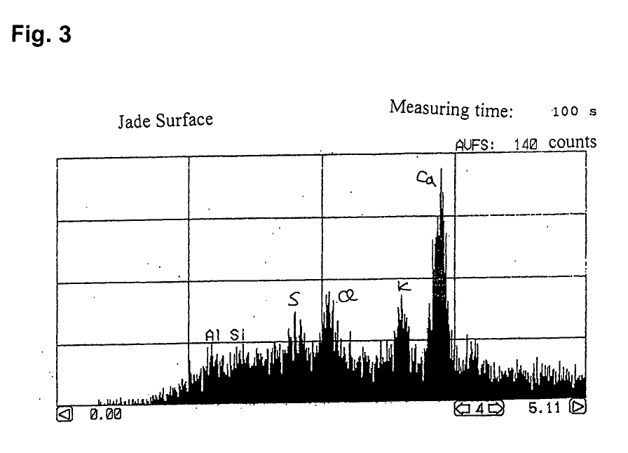

Jade was mixed in the feed and fed to the silkworms. The silk they produced was inspected using an energy dispersive X-ray analyzer. The results are shown in FIG. 3. As is clear from the figure, the jade was transferred to the silk.

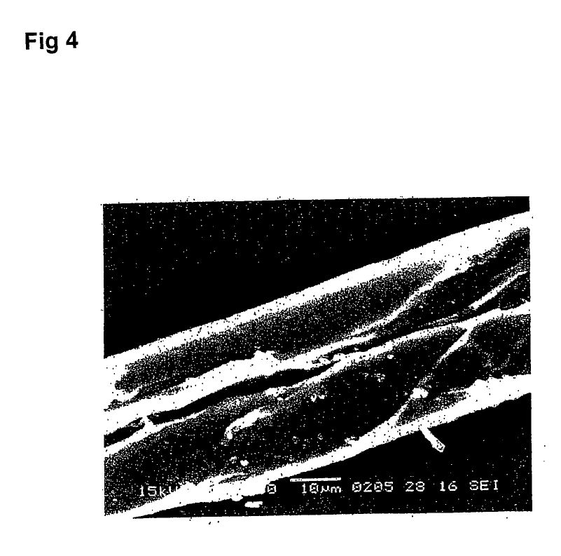

FIG. 4 is a photograph showing zeolite particles that were transferred to the cocoon filaments.

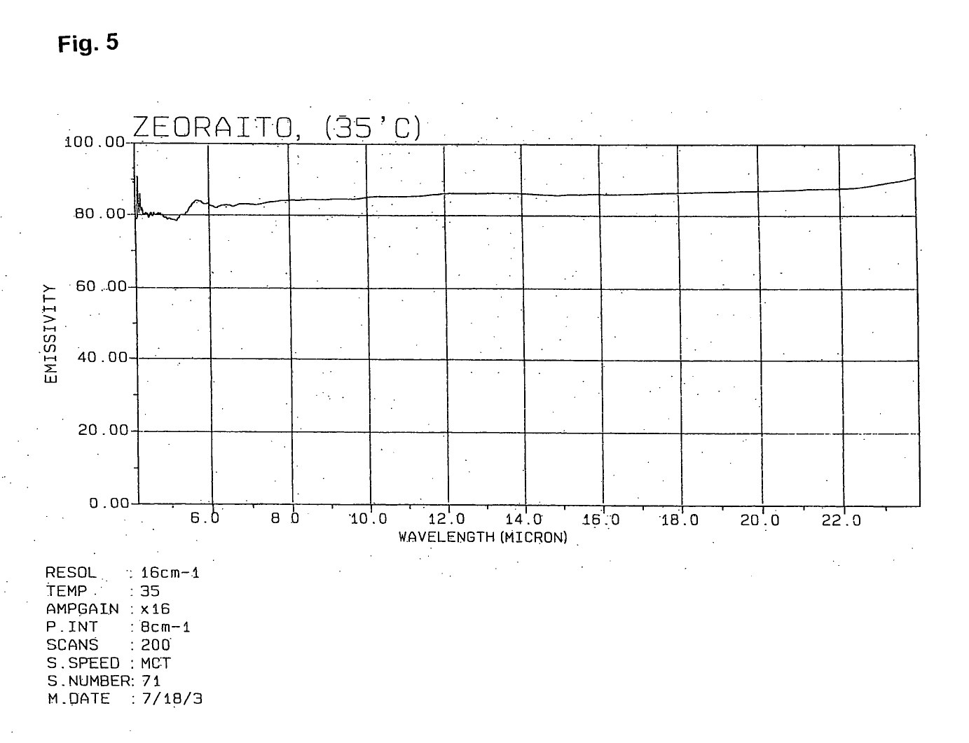

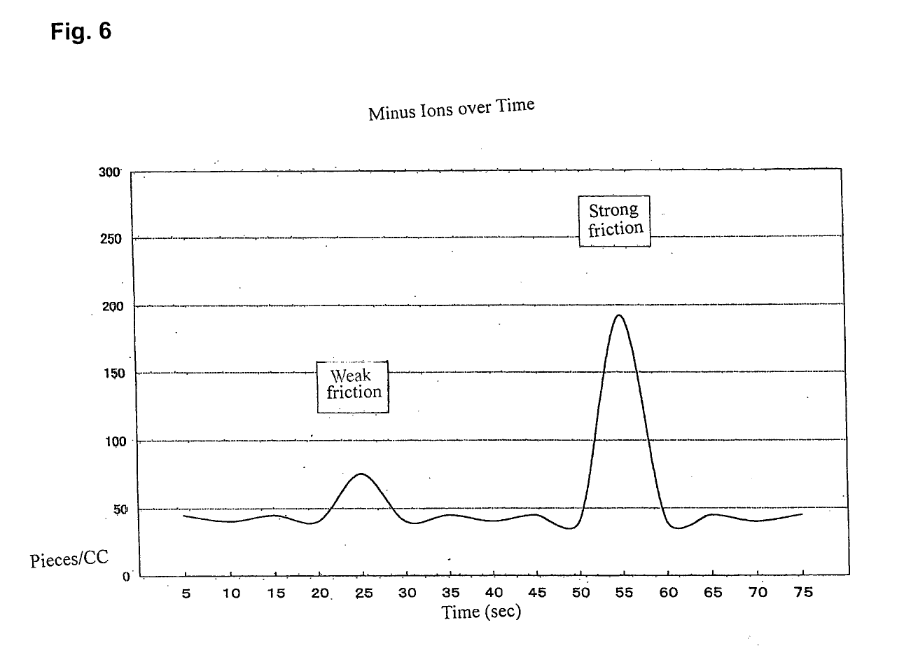

FIG. 5 shows the far-infrared emissivity of a knit product (bed sheet) made from silk containing zeolite and wool. As is clear from the figure, a product made from silk containing zeolite particles emits far-infrared rays. The graph in FIG. 6 shows minus ions emitted from a knit product (wristband) made from silk containing tourmaline and cotton.

The first method of mixing functional particles with silkworm feed and the second method of mixing aqueous solution (including distilled water) of functional particles with the silkworm feed were separately described in the above paragraphs. Both methods may be used in combination to produce the same effect. Furthermore, the silk products as used in this specification refer to all products made from silk that are currently manufactured, including various types of clothing, small articles and non-clothing items. The present invention may be implemented in various other forms of embodiment without deviating from the spirit of the main features of the present invention. The above-mentioned embodiments are therefore only a few examples and should not be construed as limiting.

Clothing made from cocoon filaments manufactured according to the present invention includes functional particles that can provide effective benefits for the human body. It is possible to manufacture a variety of clothing types containing functional particles. It is also possible to produce silk containing functional particles that is very close in texture and feeling to the charm of natural silk. The present invention is not limited to clothing but may be applied to various products. New products with unique functions may be produced by knitting or weaving silk together with other fibers.

Although only some exemplary embodiments of this invention have been described in detail above, those skilled in the art will readily appreciated that many modifications are possible in the exemplary embodiments without materially departing from the novel teachings and advantages of this invention. Accordingly, all such modifications are intended to be included within the scope of this invention.

1. A feed for a silkworm comprising a silkworm feed; and at least one of a pre-determined quantity of functional particles, an aqueous solution of functional particles of a pre-determined concentration and a distillate.

2. The feed as claimed in claim 1, wherein said silkworm feed is an artificial silkworm feed.

3. The feed as claimed in claim 2, wherein said artificial silkworm feed is KUWANOHANA.

4. The feed as claimed in claim 2, wherein said aqueous solution of functional particles contains at least 0.5 to 40% of the functional particles by weight.

5. The feed as claimed in claim 2, wherein said artificial silkworm feed contains at least 1 to 30% of the functional particles by weight.

6. The feed as claimed in claim 1, wherein said functional particles are alumina, silica, titanium oxide, zeolite, fluorite (luminous rock), tourmaline, jade, pigment, dye, temperature-sensitive pigment, UV-sensitive pigment or any other functional pigments, perfume, charcoal, cholesterol or other inorganic or organic substance.

7. The feed as claimed in claim 6, wherein said functional particles are powder particles of 10 μm or less in diameter.

8. The feed as claimed in claim 1, wherein said silkworm feed is fresh mulberry leaves.

9. The feed as claimed in claim 8, wherein said aqueous solution of functional particles contains at least 0.5 to 40% of the functional particles by weight.

10. The feed as claimed in claim 8, wherein said functional particles are alumina, silica, titanium oxide, zeolite, fluorite (luminous rock), tourmaline, jade, pigment, dye, temperature-sensitive pigment, UV-sensitive pigment or any other functional pigments, perfume, charcoal, or cholesterol or other organic or inorganic substances.

11. The feed as claimed in claim 10, wherein said functional particles are powder particles of 10 μm or less in diameter.

12. The feed as claimed in claim 1, wherein said distillate is a bamboo vinegar.

13. Silk produced by silkworms that fed on the feed claimed in any one of claims 1-12.

14. A silk product comprising the silk claimed in claim 13.
